(19) United States
(12) Patent Application Publication
(10) Pub. No.: US 2004/0180021 A1
(43) Pub. Date: Sep. 16, 2004

(54) Composition comprising at least one film-forming polymer

(76) Inventor: Valerie De La Poterie, Ie Chatelet en Brie (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/734,264

(22) Filed: Dec. 15, 2003

(30) Foreign Application Priority Data
Dec. 13, 2002 (FR) 02 15871

(51) Int. Cl. A61K 7/06; A61K 7/11

(52) U.S. Cl. 424/70.12

(57) ABSTRACT

Disclosed herein is a cosmetic composition for coating keratin fibres, comprising, in a physiologically acceptable medium:

a dispersing phase comprising an aqueous phase, at least one film-forming polymer in the form of solid particles dispersed in the aqueous phase, and at least one thickener for the aqueous phase in a sufficient amount such that the dispersing phase has a viscosity of greater than or equal to 0.2 Pa.s, and

a fatty phase with a viscosity of greater than or equal to 0.2 Pa.s, dispersed in the aqueous phase,

wherein the composition does not comprise a surfactant to disperse the fatty phase in the dispersing phase.

Disclosed herein is a cosmetic composition for coating eyelashes, comprising, in a physiologically acceptable medium, a dispersing phase comprising an aqueous phase, at least one film-forming polymer in the form of solid particles dispersed in the aqueous phase, and at least one thickener for said aqueous phase in a sufficient amount such that the dispersing phase has a viscosity of greater than or equal to 0.2 Pa.s, and a fatty phase, with a viscosity of greater than or equal to 0.2 Pa.s dispersed in the aqueous phase. The disclosed composition does not contain a surfactant sufficient to disperse the fatty phase in the dispersing phase and may in fact contain no surfactant. Further disclosed herein is a makeup or care process for a keratin material, comprising applying to the keratin material the cosmetic composition disclosed herein.

The composition may be a makeup composition, such as a mascara, a makeup base for keratin fibres, or basecoat, a composition to be applied onto a makeup, or topcoat, or alternatively a treatment composition for the eyelashes. In one embodiment, the composition is a mascara.

Document WO-A-95/15741 discloses mascara compositions in the form of wax-in-water emulsions comprising surfactants. However, on account of the presence of these surfactants, the film of makeup obtained with these compositions may not show good water resistance or resistance to rubbing, and, on contact with water (for example during bathing or showering), the film can become partially disintegrated by wearing away, or may spread around the eye. The wearing-away of the film can result in a substantial loss of intensity of the colour of the makeup, possibly obliging the consumer to reapply the mascara. The spreading of the film may also form a very unattractive aureole around the made-up area. Tears and perspiration can also give rise to these same drawbacks.

To promote the water-fastness of the makeup, it is known practice from document U.S. Pat. No. 4,423,031 to use acrylic polymers in aqueous dispersion. However, when the mascara composition comprises waxes emulsified in the aqueous phase, the makeup deposit may not show sufficient water resistance, due to the presence of surfactants, and can become disintegrated in the same way i.e., on contact with water or by rubbing.

It is also known practice to increase the solids content (comprising mainly of the fatty phase, such as the waxes) in a mascara composition in order to obtain a more substantial deposit of makeup on the eyelash, and thus a “charging” makeup result. However, the use of polymers in aqueous dispersion may not allow a charging makeup result to be obtained on the eyelashes since the content of solid particles of the dispersions may limit the increase in solids of the dry extract in the final composition.

One aspect of the present disclosure is to provide a mascara composition that allows a thick (charging) makeup result that is resistant to cold water to be obtained on the eyelashes.

The present inventor has discovered that such a mascara can be obtained by using a dispersing phase comprising an aqueous phase and at least one film-forming polymer dispersed in the aqueous phase, wherein the aqueous phase, for example through sufficient gellation, allows a fatty phase, such as at least one wax, to be dispersed in the dispersing phase without using a surfactant sufficient to accomplish the dispersion, thus making it possible to substantially increase the solids content in the mascara while at the same time conserving its water-resistance properties provided by the film-forming polymer.

The composition disclosed herein does not contain any surfactant sufficient to disperse all the fatty phase (i.e. each and every component of the fatty phase) in the dispersing phase, and of course, may contain no surfactant at all. For example, when the fatty phase comprises several waxes, the composition is such that it does not contain any surfactant sufficient to disperse each of the waxes in the dispersing phase and may in fact contain no surfactant.

After applying the composition to the eyelashes, the mascara obtained can show at least one of the following properties: good resistance to cold water, i.e. water of less than or equal to 30° C., for example, during bathing, good resistance to tears and/or perspiration, and good resistance to rubbing, such as rubbing with the fingers. Furthermore, the makeup obtained can give good thickening of the eyelashes and can thus be a good charging makeup.

Another aspect of the disclosure is a composition for coating the eyelashes, comprising, in a physiologically acceptable medium:

a dispersing phase comprising an aqueous phase, at least one film-forming polymer in the form of solid particles dispersed in the aqueous phase, and at least one thickener for the aqueous phase in a sufficient amount such that the dispersing phase has a viscosity of greater than or equal to 0.2 Pa.s, and

a fatty phase with a viscosity of greater than or equal to 0.2 Pa.s, dispersed in the aqueous phase,

wherein the composition does not comprise a surfactant sufficient to disperse the fatty phase in the dispersing phase.

The term “fatty phase” means, as disclosed herein, a phase comprising one or more non-aqueous components generally compatible with each other.

Further disclosed herein is the use, in a cosmetic composition for coating keratin fibres, comprising a physiologically acceptable medium, of the combination

of a dispersing phase comprising an aqueous phase, at least one film-forming polymer in the form of solid particles dispersed in the aqueous phase, and at least one thickener for the aqueous phase in a sufficient amount such that the dispersing phase has a viscosity of greater than or equal to 0.2 Pa.s, and

a fatty phase with a viscosity of greater than or equal to 0.2 Pa.s dispersed in the aqueous phase,

wherein the composition does not comprise a surfactant sufficient to disperse the fatty phase in the dispersing phase,

to obtain a smooth and uniform film deposited on keratin fibres, and/or a film that has at least one of the following properties: being resistant to at least one of water, tears and perspiration, and having a charging effect.

Disclosed herein is also a non-therapeutic cosmetic care or makeup process for a keratin material, comprising applying to the keratin material a composition as defined above.

Fatty Phase:

The fatty phase present in the composition has a viscosity, at room temperature (25° C.), of greater than or equal to 0.2 Pa.s, such as greater than or equal to 1 Pa.s, further such as greater than or equal to 10 Pa.s. The upper limit of the viscosity is reached when the fatty phase is in solid form and therefore does not have a measurable viscosity.

The viscosity is measured using a Rheomat RM 180 viscometer equipped with an MS-r3 or MS-r4 spindle rotating at either 240 min⁻¹ for a 60 Hz power supply or at 200 min⁻¹ for a 50 Hz power supply.

The fatty phase may comprise at least one entity chosen from waxes, gums, pasty fatty substances, semi-crystalline polymers, oils, and oils thickened with at least one structuring agent.

As disclosed herein, a wax is a lipophilic compound, which is solid at ambient temperature (25° C.), undergoes a reversible solid/liquid change of state, and may have a melting point ranging from 30° C. to 120° C. By bringing the wax to the liquid state (melting), it is possible to make it miscible with oils and to form a microscopically homogeneous mixture, but on returning the temperature of the mixture to the ambient temperature, a recrystallization of the wax in the oils of the mixture can be obtained.

The melting point of the wax may be measured using a differential scanning calorimeter (DSC), for example, the calorimeter sold under the name DSC 30 by the company Metler. A sample of a 15 mg product placed in a crucible is heated from 0C to 120° C., at a heating rate of 10° C./minute, is then cooled from 120° C. to 0° C. at a cooling rate of 10° C./minute and is then finally reheated from 0° C. to 120° C. at a heating rate of 5° C./minute. During the reheating, the variation in the difference in power absorbed by the empty crucible and by the crucible containing the sample product is measured as a function of the temperature. The melting point of the product is the value of the temperature corresponding to the top of the peak of the curve representing the variation in the difference in power absorbed as a function of the temperature.

The waxes that may be used in the composition may be chosen from waxes that are solid and rigid at room temperature, of animal, mineral, plant or synthetic origin, and mixtures thereof. The waxes may have a melting point ranging, for example, from 30° C. to 120° C., such as from 30° C. to 70° C. The wax may also have a hardness ranging, for example, from 0.5 MPa to 15 MPa, such as from 3 MPa to 15 MPa. The hardness is determined by measuring the compressive force, measured at 20° C. using a texturometer sold under the name TA-TX2i by the company Rheo, equipped with a stainless steel cylinder 2 mm in diameter travelling at a measuring speed of 0.1 mm/s, and penetrating into the wax to a penetration depth of 0.3 mm. To carry out the hardness measurement, the wax is melted at a temperature equal to the melting point of the wax +20° C. The molten wax is poured into a container with 30 mm in diameter and 20 mm deep. The wax is recrystallized at ambient temperature (25° C.) over 24 hours and the wax is then stored for at least 1 hour at 20° C. before carrying out the hardness measurement. The hardness value is the compressive force measured divided by the area of the texturometer cylinder in contact with the wax.

For example, a polar wax may be used. The term “polar wax” means a wax comprising chemical compounds comprising at least one polar group. Polar groups are well known to those skilled in the art and may be chosen, for example, from alcohol, ester and carboxylic acid groups. Polyethylene waxes, paraffin waxes, microcrystalline waxes, ozokerite and Fisher-Tropsch waxes are not polar waxes.

For example, the polar waxes can have a mean Hansen solubility parameter δₐ at 25° C. such that δₐ>0 (J/cm³)^{1/2}, for example, δₐ>1 (J/cm³)^{1/2},

δₐ=\sqrt{δₚ²+δₕ²}

in which δₚ and δₕ are, respectively, the contributions of polar type and of specific interaction type in the Hansen solubility parameters.

The definition of solvents in the three-dimensional solubility space according to Hansen is described in the article by C. M. Hansen: “The three-dimensional solubility parameters” J. Paint Technol. 39, 105 (1967);

δₕ characterizes the specific interaction forces (such as hydrogen bonding, acid/base, donor/acceptor, etc. interactions);

δₚ characterizes the Debye interaction forces between permanent dipoles and the Keesom interactions forces between induced dipoles and permanent dipoles.

The parameters δₚ and δₕ are expressed in (J/cm³)^{1/2}.

Non-limiting examples of the waxes that may be used include beeswax, lanolin wax, Chinese insect waxes, candelilla wax, ouricury wax, cork fibre wax, sugarcane wax, berry wax, Japan wax and sumach wax; montan wax, waxy copolymers, and also esters thereof.

Mention may also be made of the waxes obtained by catalytic hydrogenation of animal or plant oils comprising at least one fatty chain chosen from linear and branched C₈-C₃₂ fatty chains. Among these, mention may be made, for example, of hydrogenated jojoba oil, hydrogenated sunflower oil, hydrogenated castor oil, hydrogenated coconut oil, hydrogenated lanolin oil, and olive wax obtained by hydrogenation of olive oil esterified with stearyl alcohol, such as the Phytowax Olive 18 L57 from the company Sophim.

Mention may also be made of silicone waxes and fluoro waxes.

For instance, beeswax, candelilla wax, berry wax, hydrogenated jojoba wax, olive wax obtained by hydrogenation of olive oil esterified with stearyl alcohol, and mixtures thereof, may also be used.

The gums used in the composition are fatty substances in the form of polymers that are solid at room temperature, with a weight-average molecular weight ranging from 50,000 to 1,000,000. The gum is often sold as a dispersion in an organic solvent, such as silicone oil.

The gum may be, for example, a silicone gum corresponding to the formula:

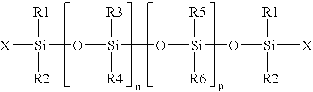

wherein:

R₁, R₂, R₅ and R₆, which may be identical or different, are chosen from alkyl radicals comprising from 1 to 6 carbon atoms,

R₃ and R₄, which may be identical or different, are chosen from alkyl radicals comprising from 1 to 6 carbon atoms and aryl radicals,

X, which may be identical or different, is chosen from alkyl radicals comprising from 1 to 6 carbon atoms, a hydroxyl radical and a vinyl radical, and

n and p, which may be identical or different, are chosen so as to give the silicone gum a viscosity, at room temperature (25° C.), of greater than 100,000 mPa.s, for example, greater than 500,000 mPa.s.

In general, n and p can each range from 0 to 5,000, for example, from 0 to 3,000.

As silicone gums that may be used herein, mention may be made of those for which:

the substituents R₁, R₂, R₃, R₄, R₅, R₆ and X are each a methyl group, p=0 and n=2,700, such as the product sold under the name SE30 by the company General Electric,

the substituents R₁, R₂, R₃, R₄, R₅, R₆ and X are each a methyl group, p=0 and n=2,300, such as the product sold under the name AK 500 000 by the company Wacker,

the substituents R₁, R₂, R₃, R₄, R₅, R₆ are each a methyl group, the substituent X is a hydroxyl group, p=0 and n=2,700, as a 13% solution in cyclopentasiloxane, such as the product sold under the name Q2-1401 by the company Dow Corning,

the substituents R₁, R₂, R₃, R₄, R₅, R₆ are each a methyl group, the substituent X is a hydroxyl group, p=0 and n=2,700, as a 13% solution in polydimethylsiloxane, such as the product sold under the name Q2-1403 by the company Dow Corning, and

the substituents R₁, R₂, R₅, R₆ and X are each a methyl group and the substituents R₃ and R₄, are each an aryl group, such that the molecular weight of the silicone gum is about 600,000, for instance the product sold under the name 761 by the company Rhône-Poulenc.

The term “pasty fatty substance” means a fatty substance with a hardness, measured at room temperature as described above, ranging from 0.001 to 0.5 MPa, for example, from 0.005 to 0.4 MPa. A pasty fatty substance also has a melting point ranging from 20 to 60° C., such as from 25 to 45° C.

A pasty compound is a viscous product comprising a solid fraction and a liquid fraction.

Non-limiting examples of pasty fatty substances include polydimethylsiloxanes (PDMSs) comprising pendent chains of the alkyl or alkoxy type comprising from 8 to 24 carbon atoms, for instance stearyl dimethicone, such as those sold by Dow Corning under the references DC2503 and DC25514; esters of fatty alcohols or fatty acids comprising from 20 to 55 carbon atoms (melting point ranging, for example, from 20° C. to 35° C.), for instance cholesterol esters such as hydrogenated triglycerides of plant origin, for instance the hydrogenated castor oil sold under the name “Thixinr” by the company Rheox, polyvinyl laurate, arachidyl propionate, triisostearyl or cetyl citrate, and PVP/eicosene copolymer; lanolins and derivatives thereof, for instance acetylated lanolins, oxypropylenated lanolins and isopropyl lanolate, with a melting point ranging from 25 to 45° C.; and mixtures thereof.

The fatty phase of the composition disclosed herein may comprise at least one semi-crystalline polymer.

The term “semi-crystalline polymer” means a polymer comprising a crystallizable portion such as a crystallizable pendent chain or a crystallizable block in the polymer skeleton and an amorphous portion in the polymer skeleton, and having a temperature of first-order reversible change of phase, such as the melting point (solid-liquid transition). When the crystallizable portion is in the form of a crystallizable block of the polymer skeleton, the amorphous portion of the polymer is in the form of an amorphous block; in this case, the semi-crystalline polymer is a block copolymer, chosen, for example, from diblock, triblock and multiblock type copolymers, comprising at least one crystallizable block and at least one amorphous block. The term “block” generally means at least 5 identical repeating units. The crystallizable blocks are then of different chemical nature than the amorphous blocks.

The semi-crystalline polymer disclosed herein may have a melting point of greater than or equal to 30° C., such as from 30° C. to 70° C., and further such as from 30° C. to 60° C. This melting point is a temperature of first-order change of state.

The melting point may be measured by any known method such as differential scanning calorimetry (DSC).

The semi-crystalline polymer disclosed herein may, for example, have a number-average molecular mass (\overline{M}n) of greater than or equal to 1,000, ranging, for example, from 2,000 to 800,000, such as from 3,000 to 500,000, further such as from 4,000 to 150,000. The number-average molecular mass of the semi-crystalline polymer disclosed herein may, for example, be less than 100,000, such as ranging from 4,000 to 99,000. In one embodiment, the semi-crystalline polymer disclosed herein has a number-average molecular mass of greater than 5,600, for example, ranging from 5,700 to 99,000.

As used herein, the term “crystallizable chain or block” means a chain or block which, if it were obtained alone, could change from the amorphous state to the crystalline state reversibly, depending on whether one is above or below the melting point. As disclosed herein, a “chain” is a group of atoms, which are pendent or lateral relative to the polymer skeleton. A “block” is a group of atoms belonging to the skeleton, wherein this group constitutes one of the repeating units of the polymer. For instance, the “crystallizable pendent chain” may be a chain comprising at least 6 carbon atoms.

The crystallizable block or blocks of the semi-crystalline polymers may present in an amount of, for example, at least 30% of the total weight of each polymer, such as at least 40%. The semi-crystalline polymers disclosed herein comprising crystallizable blocks may be block or multiblock polymers. They may be obtained by polymerizing a monomer comprising at least one reactive or ethylenic double bond or by polycondensation. When the semi-crystalline polymers disclosed herein are polymers containing crystallizable pendent chains, these pendent chains may be, for example, in random or statistical form.

The semi-crystalline polymers disclosed herein may be, for example, of synthetic origin. Furthermore, they do not comprise a polysaccharide skeleton. In general, the crystallizable units (chains or blocks) of the semi-crystalline polymers disclosed herein may be derived from monomers comprising crystallizable blocks or chains, used for the manufacture of the semi-crystalline polymers.

Non-limiting examples of the semi-crystalline polymers disclosed herein include:

block copolymers of polyolefins of controlled crystallization, such as those whose monomers are described in EP-A-0 951 897,

polycondensates, such as those chosen from aliphatic polyester types, aromatic polyester types andaliphatic/aromatic copolyester types,

homopolymers or copolymers bearing at least one crystallizable hydrophobic pendent chain and homopolymers or copolymers bearing in the skeleton at least one crystallizable block, for instance those described in document U.S. Pat. No. 5,156,911,

homopolymers or copolymers bearing at least one crystallizable hydrophobic pendent chain, for example, bearing at least one fluoro group, as described in document WO-A-01/1 9333,

and mixtures thereof.

A) Semi-Crystalline Polymers Comprising at Least One Crystallizable Pendent Chain

Mention may be made, for example, of those semi-crystalline polymers defined in documents U.S. Pat. No. 5,156,911 and WO-A-01/19333. They are homopolymers or copolymers comprising from 50% to 100% by weight of units resulting from the polymerization of at least one monomer bearing a crystallizable hydrophobic pendent chain.

These homopolymers or copolymers may be of any nature, provided that they meet the conditions mentioned previously.

These homopolymers or copolymers can result:

from the polymerization, such as the free-radical polymerization, of at least one monomer comprising at least one double bond chosen from reactive and ethylenic double bonds with respect to a polymerization, such as a vinyl, (meth)acrylic or allylic group, and

from the polycondensation of at least one monomer comprising at least one co-reactive group chosen, for example, from carboxylic acid, sulphonic acid, alcohol, amine and isocyanate, such as polyesters, polyurethanes, polyethers, polyureas and polyamides.

In general, these polymers may be chosen from, for example, homopolymers and copolymers resulting from the polymerization of at least one monomer comprising at least one crystallizable chain of formula X:

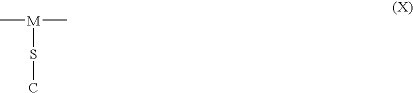

wherein M is an atom of the polymer skeleton, S is a spacer and C is a crystallizable group.

The crystallizable chains “—S—C” may be aliphatic or aromatic, and optionally fluorinated or perfluorinated. “S”, for example, may be a group chosen from (CH₂)ₘ, (CH₂CH₂O)ₘ, and (CH₂O), which may be linear or branched or cyclic, wherein m is an integer ranging from 0 to 22. “S” may, for example, be a linear group. “S” and “C” may be different.

When the crystallizable chains “—S—C” are hydrocarbon-based aliphatic chains, they comprise hydrocarbon-based alkyl chains comprising at least 11 carbon atoms and not more than 40 carbon atoms, such as 24 carbon atoms. They may be, for example, aliphatic chains or alkyl chains comprising at least 12 carbon atoms, for example, C₁₄-C₂₄ alkyl chains. When they are fluoroalkyl or perfluoroalkyl chains, they comprise at least six fluorinated carbon atoms, for example, among at least 11 carbon atoms, at least six of which are fluorinated.

Non-limiting examples of the semi-crystalline polymers or copolymers comprising at least one crystallizable chain include those resulting from the polymerization of at least one monomer chosen from: (meth)acrylates of saturated alkyls comprising at least one C₁₄-C₂₄ group; perfluoroalkyl (meth)acrylates comprising at least one C₁₁-C₁₅ perfluoroalkyl group; N-alkyl(meth)acrylamides comprising at least one C₁₄-C₂₄ alkyl group (with or without a fluorine atom); vinyl esters comprising alkyl or perfluoro(alkyl) chains comprising at least one C₁₄-C₂₄ alkyl group (comprising at least 6 fluorine atoms per perfluoroalkyl chain); vinyl ethers comprising alkyl or perfluoro(alkyl) chains comprising at least one C₁₄-C₂₄ alkyl group (comprising at least 6 fluorine atoms per perfluoroalkyl chain); C₁₄-C₂₄ alpha-olefins such as octadecene, para-alkylstyrenes comprising at least one C₁₂-C₂₄ alkyl group; and mixtures thereof.

When the semi-crystalline polymers result from a polycondensation, the hydrocarbon-based and/or fluorinated crystallizable chains as defined above are borne by a monomer chosen from a diacid, a diol, a diamine and a diisocyanate.

When the semi-crystalline polymers disclosed herein are copolymers, they further comprise from 0 to 50% of groups Y and Z resulting from the copolymerization: a) of at least one Y monomer chosen from polar and non-polar monomers:

When Y is a polar monomer, it may be chosen from a monomer comprising at least one group chosen from polyoxyalkylenated groups (such as oxyethylenated and oxypropylenated groups), a hydroxyalkyl (meth)acrylate, for instance, hydroxyethyl acrylate, (meth)acrylamide, an N-alkyl(meth)acrylamide, an N,N-dialkyl(meth)acrylamide such as N,N-diisopropylacrylamide or N-vinylpyrrolidone (NVP), N-vinylcaprolactam, a monomer comprising at least one carboxylic acid group, for instance (meth)acrylic acid, crotonic acid, itaconic acid, maleic acid or fumaric acid, a monomer comprising at least one carboxylic acid anhydride group, for instance, maleic anhydride, and mixtures thereof.

When Y is a non-polar monomer, it may be chosen from an ester of the linear, branched or cyclic alkyl (meth)acrylate type, a vinyl ester, an alkyl vinyl ether, an alpha-olefin, a styrene, a styrene substituted with a C₁ to C₁₀ alkyl group, for instance α-methylstyrene, and a macromonomer of the polyorganosiloxane type comprising at least one vinyl unsaturation.

As disclosed herein, the term “alkyl” means a saturated group of, for example, C₈ to C₂₄, such as C₁₄ to C₂₄, except where otherwise mentioned; and b) of at least one Z polar monomer, wherein Z has the same definition as the “polar Y” defined above.

The semi-crystalline polymers comprising at least one crystallizable pendent chain may be chosen from, for example, alkyl (meth)acrylate and alkyl(meth)acrylamide homopolymers comprising at least one alkyl group as defined above, such as at least one C₁₄-C₂₄ alkyl group, copolymers of these monomers with at least one hydrophilic monomer, for example, of different nature from (meth)acrylic acid, such as N-vinylpyrrolidone and hydroxyethyl (meth)acrylate, and mixtures thereof.

B) Polymers Bearing in the Skeleton at Least One Crystallizable Block

These polymers may be, for example, block copolymers comprising at least two blocks of different chemical nature, one of which is crystallizable.

The block polymers defined in U.S. Pat. No. 5,156,911 may be used;

Block copolymers of olefin or of cycloolefin comprising at least one crystallizable chain, for instance those derived from the block polymerization of at least one monomer chosen from

cyclobutene, cyclohexene, cyclooctene, norbornene (i.e. bicyclo(2,2,1)-2-heptene), 5-methylnorbornene, 5-ethyinorbornene, 5,6-dimethyinorbornene, 5,5,6-trimethyinorbomene, 5-ethylidenenorbornene, 5-phenylnorbornene, 5-benzylnorbornene, 5-vinyinorbornene, 1,4,5,8-dimethano-1,2,3,4,4a,5,8a-octahydronaphthalene, and dicyclopentadiene,

with at least one monomer chosen from

ethylene, propylene, 1-butene, 3-methyl-1 -butene, 1-hexene, 4-methyl-1-pentene, 1-octene, 1-decene and 1-eicosene,

and such as copoly(ethylene/norbornene) blocks and (ethylene/propylene/ethylidene-norbornene) block terpolymers. Those resulting from the block copolymerization of at least two C₂-C₁₆, such as C₂-C₁₂, further such as C₄-C₁₂ α-olefins such as those mentioned above, for example, block bipolymers of ethylene and of 1-octene may also be used.

The copolymers may be copolymers comprising at least one crystallizable block, wherein the copolymer residue is amorphous (at room temperature). These copolymers may also comprise two crystallizable blocks of different chemical nature. The copolymers as disclosed herein, may be chosen, for example, from those that simultaneously comprise, at room temperature, a crystallizable block and an amorphous block that are both hydrophobic and lipophilic, sequentially distributed; mention may be made, for example, of polymers comprising one of the crystallizable blocks and one of the amorphous blocks below:

Blocks that may be crystallizable by nature: a) polyester, for instance poly(alkylene terephthalate), b) polyolefin, for instance polyethylenes and polypropylenes.

Amorphous and lipophilic blocks, for instance amorphous polyolefins and copoly(olefin)s, such as poly(isobutylene), hydrogenated polybutadiene and hydrogenated poly(isoprene).

Non-limiting examples of such copolymers comprising a crystallizable block and a separate amorphous block include:

A) poly(ε-caprolactone)-b-poly(butadiene) block copolymers, for example, those which are hydrogenated, such as those described in the article “Melting behavior of poly(ε-caprolactone)-block-polybutadiene copolymers” from S. Nojima, Macromolecules, 32, 3727-3734 (1999),

B) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

C) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997), and

D) the poly(ethylene)-b-poly(ethylethylene) block copolymers cited in the general article “Crystallization in block copolymers” by I. W. Hamley, Advances in Polymer Science, Vol.148, 113-137 (1999).

The semi-crystalline polymers in the composition disclosed herein may be partially crosslinked, provided that the degree of crosslinking does not interfere with their dissolution or dispersion in the liquid fatty phase by heating above their melting point. It may then be a chemical crosslinking, by reaction with a multifunctional monomer during the polymerization. It may also be a physical crosslinking which may, in this case, be due either to the establishment of bonds of hydrogen or dipolar type between groups borne by the polymer, such as the dipolar interactions between carboxylate ionomers, these interactions being of small amount and borne by the polymer skeleton; or to a phase separation between the crystallizable blocks and the amorphous blocks borne by the polymer.

The semi-crystalline polymers in the composition may be, for example, non-crosslinked.

According to one embodiment, the semi-crystalline polymer is chosen from copolymers resulting from the polymerization of at least one monomer comprising a crystallizable chain chosen from saturated C₁₄ to C₂₄ alkyl (meth)acrylates, C₁ to C₁₅ perfluoroalkyl (meth)acrylates, C₁₄ to C₂₄ N-alkyl(meth)acrylamides with or without a fluorine atom, vinyl esters comprising at least one chain chosen from C₁₄ to C₂₄ alkyl and perfluoroalkyl chains, vinyl ethers comprising at least one chain chosen from C₁₄ to C₂₄ alkyl and perfluoroalkyl chains, C₁₄ to C₂₄ alpha-olefins, para-alkylstyrenes with an alkyl group comprising from 12 to 24 carbon atoms, with at least one monomer chosen from optionally fluorinated C₁ to C₁₀ monocarboxylic acid esters and amides of the following formula:

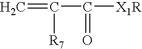

wherein R₇ is chosen from H and CH₃, R is a group chosen from optionally fluorinated C₁-C₁₀ alkyl groups and X, is chosen from O, NH and NR₈ wherein R₈ is a group chosen from optionally fluorinated C₁-C₁₀ alkyl groups.

According to one embodiment disclosed herein, the semi-crystalline polymer is derived from a monomer comprising a crystallizable chain, chosen from saturated C₁₄ to C₂₂ alkyl (meth)acrylates.

As non-limiting examples of the structuring semi-crystalline polymers that may be used in the composition disclosed herein, mention may be made of the products Intelimer® from the company Landec, described in the brochure “Intelimer® polymers”, Landec IP22 (Rev. 4-97). These polymers are in solid form at room temperature (25° C.). They comprise crystallizable pendent chains of the formula X above.

The semi-crystalline polymers may also be, for example, chosen from: those described in Examples 3, 4, 5, 7, 9 and 13 of U.S. Pat. No. 5,156,911 comprising a —COOH group, resulting from the copolymerization of acrylic acid and of C₅ to C₁₆ alkyl (meth)acrylate and, for example, of the copolymerization:

of acrylic acid, of hexadecyl acrylate and of isodecyl acrylate in a 1/16/3 weight ratio;

of acrylic acid and of pentadecyl acrylate in a 1/19 weight ratio;

of acrylic acid, of hexadecyl acrylate and of ethyl acrylate in a 2.5/76.5/20 weight ratio;

of acrylic acid, of hexadecyl acrylate and of methyl acrylate in a 5/85/10 weight ratio;

of acrylic acid and of octadecyl methacrylate in a 2.5/97.5 weight ratio;

of hexadecyl acrylate, of polyethylene glycol methacrylate monomethyl ether comprising 8 ethylene glycol units, and of acrylic acid in an 8.5/1/0.5 weight ratio.

It is also possible to use the structure “O” from National Starch, as described in U.S. Pat. No. 5,736,125, with a melting point of 44° C., and also semi-crystalline polymers with crystallizable pendent chains comprising fluoro groups, as described in Examples 1, 4, 6, 7 and 8 of document WO-A-01/19333.

It is also possible to use low-melting semi-crystalline polymers obtained by copolymerization of stearyl acrylate and of acrylic acid or of NVP, as described in U.S. Pat. No. 5,519,063 or EP-A-550 745 and, for example, those described in Examples 1 and 2 below, for the preparation of polymers, with a melting point of 40° C. and 38° C., respectively.

It is also possible to use semi-crystalline polymers obtained by copolymerization of behenyl acrylate and of acrylic acid or of NVP, as described in U.S. Pat. No. 5,519,063 and EP-A-550 745, for the preparation of polymers, with a melting point of 60° C. and 58° C., respectively.

The semi-crystalline polymers may not, for example, comprise carboxylic groups.

The fatty phase may also comprise at least one oil chosen from oils and oils thickened with at least one structuring agent chosen from lipophilic gelling agents, and organogelling agents, such that this fatty phase has a viscosity of greater than or equal to 0.2 Pa.s.

The term “oil” means a fatty substance that is liquid at room temperature (25° C.) and atmospheric pressure (760 mmHg, i.e. 105 Pa).

The oil may be chosen from any physiologically acceptable such as cosmetically acceptable oils, for example, mineral, animal, plant and synthetic oils; such as volatile or non-volatile hydrocarbon-based and/or silicone and/or fluoro oils, and mixtures thereof. The term “hydrocarbon-based oil” means an oil mainly comprising carbon and hydrogen atoms and possibly at least one functional group chosen from hydroxyl, ester, ether and carboxylic functional groups. In general, the oil has a viscosity, at room temperature (25° C.) ranging from 0.5 to 100,000 mPa.s, such as from 50 to 50,000 mPa.s, further such as from 100 to 300,000 mPa.s.

Non-limiting examples of oils that may be used herein include:

hydrocarbon-based oils of animal origin such as perhydrosqualene;

hydrocarbon-based plant oils such as liquid triglycerides of fatty acids of 4 to 24 carbon atoms, for instance heptanoic or octanoic acid triglyceride, or alternatively sunflower oil, maize oil, soybean oil, marrow oil, grapeseed oil, sesame seed oil, hazelnut oil, apricot oil, macadamia oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter;

linear or branched hydrocarbons of mineral or synthetic origin, such as liquid paraffins and derivatives thereof, petroleum jelly, polydecenes, polybutenes and hydrogenated polyisobutene such as parleam;

synthetic esters and ethers, such as those of fatty acids, for instance the oils of formula R₉COOR₁₀ wherein R₉ is a higher fatty acid residue comprising from 1 to 40 carbon atoms and R₁₀ is a hydrocarbon-based chain comprising from 1 to 40 carbon atoms with R₉+R₁₀>10, for instance purcellin oil, isononyl isononanoate, isopropyl myristate, 2-ethylhexyl palmitate, 2-octydodecyl stearate, 2-octyldocecyl erucate, isostearyl isostearate or tridecyl trimellitate; hydroxylated esters, for instance isostearyl lactate, octyl hydroxystearate, octyldodecyl hydroxystearate, diisostearyl malate, triisocetyl citrate or fatty alkyl heptanoates, octanoates or decanoates; polyol esters, for instance propylene glycol dioctanoate, neopentyl glycol diheptanoate or diethylene glycol diisononanoate; and pentaerythritol esters, for instance pentaerythrityl tetraisostearate;

fatty alcohols comprising from 12 to 26 carbon atoms, for instance octyidodecanol, 2-butyloctanol, 2-hexyldecanol, 2-undecylpentadecanol or oleyl alcohol;

fluoro oils, which are optionally partially hydrocarbon-based and/or silicone-based;

silicone oils, for instance volatile or non-volatile, linear or cyclic polydimethylsiloxanes (PDMSs); polydimethylsiloxanes comprising alkyl, alkoxy or phenyl groups, pendent or at the end of a silicone chain, these groups comprising from 2 to 24 carbon atoms; phenyl silicones, for instance phenyl trimethicones, phenyl dimethicones, phenyl trimethylsiloxy diphenyl siloxanes, diphenyl dimethicones, diphenyl methyldiphenyl trisiloxanes and 2-phenyl ethyl trimethyl siloxysilicates; and

mixtures thereof.

The at least one structuring agent may be chosen, for example, from lipophilic gelling agents and organogelling agents.

The lipophilic gelling agent may be organic or mineral, and polymeric or molecular.

Mineral lipophilic gelling agents that may be mentioned include optionally modified clays, for instance hectorites modified with a C₁₀ to C₂₂ fatty acid ammonium chloride, for instance hectorite modified with distearyidimethylammonium chloride.

Mention may also be made of fumed silica optionally subjected to a hydrophobic surface treatment, the particle size of which is less than 1 μm. It is, for example, possible to chemically modify the surface of the silica, by chemical reaction generating a reduced number of silanol groups present at the surface of the silica. It is also possible, for example, to substitute silanol groups with hydrophobic groups: a hydrophobic silica is then obtained. The hydrophobic groups may be chosen from:

trimethylsiloxyl groups, which are obtained, for example, by treating fumed silica in the presence of hexamethyldisilazane. Silicas thus treated are known as “silica silylate” according to the CTFA (6th edition, 1995). They are sold, for example, under the references “Aerosil R812®” by the company Degussa, and “Cab-O-Sil TS-530®” by the company Cabot;

dimethylsilyloxyl or polydimethylsiloxane groups, which may be obtained, for example, by treating fumed silica in the presence of polydimethylsiloxane or dimethyldichlorosilane. Silicas thus treated are known as “silica dimethyl silylate” according to the CTFA (6th edition, 1995). They are sold, for example, under the references “Aerosil R972®” and “Aerosil R974®” by the company Degussa, and “Cab-O-Sil TS-610®” and “Cab-O-Sil TS-720®” by the company Cabot.

The hydrophobic fumed silica may have, for example, a particle size that may be nanometric to micrometric, for example ranging from 5 to 200 nm.

The polymeric organic lipophilic gelling agents may be, for example, partially or totally crosslinked elastomeric organopolysiloxanes of three-dimensional structure, for instance those sold under the names KSG6, KSG16 and KSG18 from Shin-Etsu, Tefil E-505C or Trefil E-506C from Dow Corning, Gransil SR-CYC, SR DMF 10, SR-DC556, SR 5CYC gel, SR DMF 10 gel and SR DC 556 gel from Grant Industries and SF 1204 and JK 113 from General Electric; ethylcellulose, for instance those sold under the name Ethocel by Dow Chemical; polyamides, such as copolymers of a C₃₆ diacid condensed with ethylenediamine, with a weight-average molecular mass of about 6000, such as those sold by the company Arizona Chemical under the names Uniclear 80 and Uniclear 100, and galactomannans comprising from one to six, such as from two to four, hydroxyl groups per saccharide, substituted with at least one alkyl chain chosen from saturated and unsaturated alkyl chain, for instance guar gum alkylated with C₁ to C₆, such as C₁ to C₃, alkyl chains, and mixtures thereof.

Non-limiting examples of lipophilic gelling agents that are used include non-polymeric, molecular organic gelling agents, also known as organogelling agents, which are compounds whose molecules are capable of establishing between themselves physical interactions leading to self-aggregation of the molecules with formation of a supramolecular 3D network that is responsible for the gelation of the liquid fatty phase.

As disclosed herein, the term “liquid fatty phase” means a fatty phase that is liquid at room temperature (25° C.) and atmospheric pressure (760 mmHg, i.e. 105 Pa), comprising at least one fatty substance that is liquid at room temperature. These fatty substances are also known as oils, which are generally mutually compatible.

The supramolecular network may result from the formation of a network of fibrils (caused by the stacking or aggregation of organogelling molecules), which immobilizes the molecules of the liquid fatty phase.

The ability to form this network of fibrils, and thus to gel, depends on the nature (or chemical class) of the organogelling agent, on the nature of the substituents borne by its molecules for a given chemical class, and on the nature of the liquid fatty phase.

The physical interactions are of diverse nature but exclude co-crystallization. These physical interactions may be chosen, for example, from interactions of self-complementary hydrogen interaction type, π interactions between unsaturated rings, dipolar interactions, coordination bonds with organometallic derivatives, and combinations thereof. In general, each molecule of an organogelling agent can establish several types of physical interaction with a neighbouring molecule. Thus, the molecules of the organogelling agents disclosed hereinmay, for example, comprise at least one group, such as at least two groups, capable of establishing hydrogen bonds, at least one aromatic ring such as two aromatic rings, at least one ethylenically unsaturated bond and/or at least one asymmetric carbon. The groups capable of forming hydrogen bonds may be chosen, for example, from hydroxyl, carbonyl, amine, carboxylic acid, amide, urea and benzyl groups, and combinations thereof.

The organogelling agents disclosed herein are soluble in the liquid fatty phase after heating to obtain a transparent uniform liquid phase. They may be solid or liquid at room temperature and atmospheric pressure.

The molecular organogelling agents that may be used in the composition disclosed herein may be, for example, chosen from those described in the document “Specialist Surfactants” edited by D. Robb, 1997, pp. 209-263, Chapter 8 by P. Terech, European patent applications EP-A-1 068 854 and EP-A-1 086 945, or alternatively in patent application WO-A-02/47031.

Mention may be made, for example, among these organogelling agents, of amides of carboxylic acids, such as tricarboxylic acids, for instance cyclohexanetricarboxamides (see European patent application EP-A-1 068 854), diamides with hydrocarbon-based chains each comprising from 1 to 22 carbon atoms, for example from 6 to 18 carbon atoms, wherein the hydrocarbon-based chains may be unsubstituted or substituted with at least one substituent chosen from ester, urea and fluoro groups (see patent application EP-A-1 086 945) and, for example, diamides resulting from the reaction of diaminocyclohexane, such as diaminocyclohexane in trans form, and of an acid chloride, for instance N,N′-bis(dodecanoyl)-1,2-diaminocyclohexane, N-acylamino acid amides, for instance the diamides resulting from the action of an N-acylamino acid with amines comprising from 1 to 22 carbon atoms, for instance those described in document WO-93/23008 and such as N-acylglutamic acid amides in which the acyl group is a C₈ to C₂₂ alkyl chain, such as N-lauroyl-L-glutamic acid dibutylamide, manufactured or sold by the company Ajinomoto under the name GP-1, and mixtures thereof.

The at least one structuring agent may be present in an amount ranging from 0.01 % to 90%,, for example from 0.5% to 80%, further for example, from 1 % to 70%, by weight relative to the weight of the fatty phase.

The fatty phase may be present in the composition disclosed herein in an amount ranging from 5% to 60% by weight, for example from 10% to 50% by weight, further for example, from 15% to 40% by weight, relative to the total weight of the composition.

According to one embodiment disclosed herein, the fatty phase is formed from particles of at least one wax dispersed in the aqueous phase. Thus, disclosed herein a composition for coating the eyelashes, comprising, in a physiologically acceptable medium:

a dispersing phase comprising an aqueous phase, at least one film-forming polymer in the form of solid particles dispersed in the aqueous phase, and at least one thickener for the aqueous phase in a sufficient amount such that the aqueous phase has a viscosity of greater than or equal to 0.2 Pa.s, and

at least one wax in the form of particles dispersed in the aqueous phase, wherein the composition does not comprise a surfactant sufficient to disperse the at least one wax in the dispersing phase.

Dispersing Phase:

The dispersing phase comprises an aqueous phase, at least one film-forming polymer in the form of solid particles dispersed in the aqueous phase, and at least one thickener for the aqueous phase in an amount such that the dispersing phase has a viscosity of greater than or equal to 0.2 Pa.s.

The dispersing phase of the composition may have a viscosity of greater than or equal to 0.2 Pa.s to allow the fatty phase to be dispersed without using surfactant.

The dispersing phase of the composition may have a viscosity ranging from 0.2 Pa.s to 50 Pa.s, for example, from 0.5 Pa.s to 40 Pa.s, further for example, from 1 to 30 Pa.s.

The viscosity is measured using a Rheomat RM 180 viscometer equipped with an MS-r3 or Ms-r4 spindle rotating at 240 min⁻¹ for a 60 Hz power supply or at 200 min⁻¹ for a 50 Hz power supply.

The at least one film-forming polymer is in the form of solid particles dispersed in the aqueous phase. Such a polymer dispersion is generally known as a latex or pseudolatex. The techniques for preparing these dispersions are well known to those skilled in the art.

The term “film-forming polymer” means a polymer capable of forming, by itself or in the presence of an auxiliary film-forming agent, a film that is continuous to the naked eye and that adheres to a support such as a keratin material. The auxiliary film-forming agent may be a plasticizer that is well-known to those skilled in the art.

The film-forming polymer present in the composition disclosed herein may be chosen from hydrophobic and water-soluble film-forming polymers.

The term “hydrophobic polymer” means a polymer with a solubility in water at 25° C. of less than 1% by weight.

A hydrophobic film-forming polymer, for example, may be used as film-forming polymer. A film that shows good resistance to cold water can thus be obtained.

Among the film-forming polymers which may be used in the composition disclosed herein, mention may be made of synthetic polymers, free-radical type or of polycondensate type, polymers of natural origin, and mixtures thereof.

The expression “free-radical film-forming polymer” means a polymer obtained by polymerization of monomers containing unsaturation, such as ethylenic unsaturation, wherein each monomer is capable of homopolymerizing (unlike polycondensates).

The film-forming polymers of free-radical type may be, for example, chosen from vinyl polymers and copolymers, such as acrylic polymers.

The vinyl film-forming polymers may result from the polymerization of monomers chosen from those comprising ethylenic unsaturation having at least one acid group, esters thereof, and amides thereof.

Monomers having at least one acid group that may be used may be chosen from, for example, α,β-ethylenic unsaturated carboxylic acids such as acrylic acid, methacrylic acid, crotonic acid, maleic acid and itaconic acid. In one embodiment, (meth)acrylic acid and crotonic acid are used. In another embodiment, (meth)acrylic acid is used.

The esters of the acid monomers may be chosen, for example, from the esters of (meth)acrylic acid (also known as (meth)acrylates), such as alkyl (meth)acrylates, for example, C₁-C₃₀ alkyl (meth)acrylates such as C₁-C₂₀ alkyl (meth)acrylates, aryl (meth)acrylates, for example, C₆-C₁₀ aryl (meth)acrylates, and hydroxyalkyl (meth)acrylates, such as C₂-C₆ hydroxyalkyl (meth)acrylates.

Among the alkyl (meth)acrylates which may be mentioned, for example, are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates which may be mentioned, for example, are hydroxyethyl acrylate, 2-hydroxypropyl acrylate, hydroxyethyl methacrylate and 2-hydroxypropyl methacrylate.

Among the aryl (meth)acrylates which may be mentioned, for example, are benzyl acrylate and phenyl acrylate. In one embodiment, the (meth)acrylic acid esters are chosen from alkyl (meth)acrylates.

As disclosed herein, the alkyl group of the esters may be either fluorinated or perfluorinated, i.e., some or all of the hydrogen atoms of the alkyl group are replaced with fluorine atoms.

Amides of the acid monomers which may be mentioned, for example, are (meth)acrylamides, for example, N-alkyl(meth)acrylamides, such as a C₂-C₁₂ alkyl(meth)acrylamides. Among the N-alkyl(meth)acrylamides which may be mentioned, for example, are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with at least one monomer chosen from the acid monomers, esters thereof, and amides thereof, such as those mentioned above.

Non-limiting examples of vinyl esters which may be mentioned include vinyl acetate, vinyl neodecanoate, vinyl pivalate, vinyl benzoate and vinyl t-butylbenzoate.

Styrene monomers which may be mentioned, for example, are styrene and α-methylstyrene.

The list of monomers given is not limiting, and it is possible to use any monomer known to those skilled in the art falling within the categories of acrylic and vinyl monomers, including monomers modified with at least one silicone chain.

Among the acrylic film-forming polymers disclosed herein , mention may be made of those sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® by the company Daito Kasey Kogyo, or Syntran 5760® by the company Interpolymer.

Among the film-forming polycondensates which may be mentioned are polyurethanes, polyesters, polyesteramides, polyamides and epoxyester resins, as well as polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

Among the film-forming polyurethanes disclosed herein, it is possible to use those sold under the names Neorez R-981® and Neorez R-974® by the company Avecia-Neoresins, Avalure URA405®, Avalure UR-410®, Avalure UR425®, Avalure UR-450®, Sancure 875®, Sancure 861®, Sancure 878® and Sancure 2060® by the company Goodrich, Impranil 85® by the company Bayer, and Aquamere H-1511® by the company Hydromer.

The polyesters can be obtained, in a known manner, by polycondensation of dicarboxylic acids with polyols, such as diols.

The dicarboxylic acid can be aliphatic, alicyclic or aromatic. Non-limiting examples of such acids which may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norboranedicarboxylic acid, diglycolic acid, thiodipropionic acid, 2,5-naphthalene-dicarboxylic acid and 2,6-naphthalenedicarboxylic acid. These dicarboxylic acid monomers can be used alone or in a combination of at least two dicarboxylic acid monomers. In one embodiment, at least one of phthalic acid, isophthalic acid and terephthalic acid is used.

The diol can be chosen from aliphatic, alicyclic and aromatic diols. The diols may be chosen from, for example: ethylene glycol, diethylene glycol, triethylene glycol, 1,3-propanediol, cyclohexanedimethanol, and 4-butanediol. Other polyols which can be used are glycerol, pentaerythritol, sorbitol and trimethylolpropane.

The polyesteramides can be obtained in a similar manner to that for the polyesters, by polycondensation of diacids with diamines or amino alcohols. Diamines which can be used, for example, are ethylenediamine, hexamethylenediamine and meta- or para-phenylenediamine. An amino alcohol which can be used is monoethanolamine.

The polyester can also comprise at least one monomer bearing at least one group —SO₃M, wherein M is chosen from a hydrogen atom, an ammonium ion NH₄⁺ and metal ions such as Na⁺, Li⁺, K⁺, Mg²⁺, Ca²⁺, Cu²⁺, Fe²⁺ and Fe³⁺ ions. A bifunctional aromatic monomer comprising the group —SO₃M can, for example, be used.

The aromatic nucleus of the bifunctional aromatic monomer also bearing a group —SO₃M as described above can be chosen, for example, from benzene, naphthalene, anthracene, biphenyl, oxybiphenyl, sulphonylbiphenyl and methylenebiphenyl nuclei. Non-limiting examples of bifunctional aromatic monomers also bearing a group —SO₃M which may be mentioned include: sulphoisophthalic acid, sulphoterephthalic acid, sulphophthalic acid, and 4-sulphonaphthalene-2,7-dicarboxylic acid.

Copolymers based on isophthalate/sulphoisophthalate, such as copolymers obtained by condensation of diethylene glycol, cyclohexanedimethanol, isophthalic acid and sulphoisophthalic acid, may be used. Such polymers are sold, for example, under the brand name Eastman AQ® by the company Eastman Chemical Products.

The polymers of natural origin, which are optionally modified, may be chosen from shellac resin, sandarac gum, dammar resins, elemi gums, copal resins and water-insoluble cellulose polymers, and mixtures thereof.

The particles of the at least one film-forming polymer may have a size ranging from 5 nm to 600 nm, for example, from 20 nm to 300 nm.

Aqueous dispersions of the at least one film-forming polymer that may also be used include dispersions of polymers resulting from the free-radical polymerization of at least one free-radical monomer inside and/or partially at the surface of pre-existing particles of at least one polymer chosen from polyurethanes, polyureas, polyesters, polyesteramides and alkyds. These polymers are generally known as hybrid polymers.

The at least one film-forming polymer in aqueous dispersion may be present in the composition disclosed herein in a solids (or active material) content ranging from 1% to 60% by weight relative to the total weight of the composition, for example,from 5% to 40% by weight, further for example, from 10% to 30% by weight relative to the total weight of the composition.

The composition disclosed herein may comprise at least one film-forming auxiliary agent for promoting the formation of a film with the particles of the at least one film-forming polymer. Such a film-forming agent may be chosen from any compound known to those skilled in the art as being capable of fulfilling the desired function, and, for example, may be chosen from plasticizers and coalescers.

The at least one film-forming polymer and the fatty phase may be present in a film-forming polymer/fatty phase weight ratio ranging from 0.5:1 to 1.5:1, for example, from 0.5:1 to 1.3:1.

The aqueous phase of the composition may consist essentially of water. It may also comprise a mixture of water and of at least one water-miscible solvent, for instance lower monoalcohols comprising from 1 to 5 carbon atoms, such as ethanol and isopropanol, glycols comprising from 2 to 8 carbon atoms, such as propylene glycol, ethylene glycol, 1,3-butylene glycol and dipropylene glycol, C₃-C₄ ketones and C₂-C₄ aldehydes. The aqueous phase (water and optionally the water-miscible organic solvent) is present in an amount ranging, for example, from 5% to 95% by weight relative to the total weight of the composition.

To give the dispersing phase the required viscosity, the aqueous phase of the composition comprises at least one thickener for adjusting to the desired viscosity.

Among the thickeners that may be used herein, mention may be made of:

water-soluble cellulose-based thickeners such as hydroxyethylcellulose, methylcellulose, hydroxypropylcellulose and carboxymethylcellulose. Among these, mention may be made, for example, of the gums sold under the name “Cellosize QP 4400 H” by the company Amerchol,

guar gums, for instance those sold under the name Vidogum GH 175 by the company Unipectine and under the name Jaguar C by the company Meyhall,

the quaternized guar gums sold under the name “Jaguar C-13-S” by the company Meyhall,

nonionic guar gums comprising at least one C₁-C₆ hydroxyalkyl group. Non-limiting examples of these hydroxyalkyl groups that may be mentioned include hydroxymethyl, hydroxyethyl, hydroxypropyl and hydroxybutyl groups. Such guar gums are sold, for example, under the trade names Jaguar HP8, Jaguar HP60, Jaguar HP120 and Jaguar HP 105 by the company Meyhall, or under the name Galactasol 40H4FD2 by the company Aqualon,

xanthan gum, carob gum, scleroglucan gum, gellan gum, rhamsan gum or karaya gum,

alginates, maltodextrin, starch and its derivatives, and hyaluronic acid and its salts,

clays, such as montmorillonites, hectorites and laponites,

crosslinked polyacrylic acids such as the “Carbopol” products from the company Goodrich,

polyglyceryl (meth)acrylate polymers sold under the name “Hispagel” or “Lubragel” by the companies Hispano Quimica and Guardian,

polyvinylpyrrolidone,

polyvinyl alcohol,

crosslinked acrylamide polymers and copolymers, such as those sold under the names “PAS 5161” or “Bozepol C” by the company Hoechst, and “Sepigel 305” by the company SEPPIC,

the crosslinked methacryloyoxyethyltrimethylammonium chloride homopolymers sold under the name “Salcare SC95” by the company Allied Colloid, and

associative polymers such as associative acrylic polymers.

In the composition disclosed herein, the at least one thickener for the aqueous phase may be present in an amount that is effective for the dispersing phase to have the viscosity as defined above. The amount of thickener may range, for example, from 0.1 to 15% by weight, relative to the total weight of the composition, such as from 1% to 10%, and further such as from 1% to 5% by weight, relative to the total weight of the composition.

The composition has a solids content ranging, for example, from 30% to 60% by weight relative to the total weight of the composition, further for example, from 35% to 55%, even further for example, from 40% to 50%, by weight relative to the total weight of the composition.

The solids content, i.e. the content of non-volatile material, may be measured in various ways. Non-limiting examples that may be mentioned include oven-drying methods, drying methods by exposure to infrared radiation, and chemical methods by Karl Fischer water titration.

The amount of solids, commonly referred to as “dry extract” in the compositions disclosed herein is measured, for example, by heating the sample with infrared rays with a wavelength ranging from 2 μm to 3.5 μm. The substances contained in the compositions that have a high vapour pressure evaporate under the effect of this radiation. Measuring the weight loss of the sample allows the “dry extract” of the composition to be determined. These measurements are performed using a commercial LP16 infrared desiccator from Mettler. This technique is fully described in the apparatus documentation supplied by Mettler.

The measuring protocol is as follows:

About 1 g of the composition is spread onto a metal cup. After placing this cup in the desiccator, it is subjected to infrared rays at a nominal temperature of 120° C. for one hour. The wet mass of the sample, corresponding to the initial mass, and the dry mass of the sample, corresponding to the mass after exposure to the radiation, are measured using a precision balance. The solids content (dry extract) is calculated in the following manner:

Dry extract=100×(dry mass/wet mass).

The composition disclosed herein may further comprise at least one dyestuff chosen, for example, from pulverulent dyes, liposoluble dyes and water-soluble dyes. The at least one dyestuff may be present in an amount ranging from 0.1% to 20% by weight, for example, from 1% to 15% by weight, relative to the total weight of the composition.

The pulverulent dyes may be chosen from pigments and nacreous pigments.

The pigments may be chosen from white and coloured, mineral and organic, coated and uncoated pigments. Among the mineral pigments which may be mentioned, for example, are titanium dioxide, optionally surface-treated, zirconium oxide, zinc oxide, or cerium oxide, and iron oxide or chromium oxide, manganese violet, ultramarine blue, chromium hydrate and ferric blue. Among the organic pigments which may be mentioned, for example, are carbon black, pigments of D & C type, and lacquers based on cochineal carmine or on barium, strontium, calcium or aluminium.

The nacreous pigments may be chosen from white nacreous pigments such as mica coated with titanium or with bismuth oxychloride, coloured nacreous pigments such as titanium mica with iron oxides, titanium mica with, for example ferric blue or chromium oxide, titanium mica with an organic pigment of the abovementioned type, and nacreous pigments based on bismuth oxychloride.

The liposoluble dyes may be, for example, chosen from Sudan red, D&C Red 17, D&C Green 6, β-carotene, soybean oil, Sudan brown, D&C Yellow 11, D&C Violet 2, D&C Orange 5, quinoline yellow and annatto. The water-soluble dyes may be, for example, chosen from beetroot juice and methylene blue.

The composition disclosed herein may also comprise at least one additive chosen from the additives usually used in cosmetics, such as antioxidants, preserving agents, fragrances, neutralizers, thickeners, plasticizers, cosmetic active agents, for instance emollients, moisturizers, vitamins and sunscreens, and mixtures thereof. The at least one additive may be present in the composition in an amount ranging from 0.01% to 10% by weight of the total weight of the composition.

Needless to say, a person skilled in the art will take care to select the optional additional additives and/or the amount thereof such that the advantageous properties of the composition disclosed herein are not, or are not substantially, adversely affected by the envisaged addition.

The composition disclosed herein may be manufactured by the known processes generally used in cosmetics.

The composition disclosed herein is illustrated in greater detail in the non-limiting examples that follow.

### EXAMPLE 1

A mascara having the composition below was prepared:

| | |
|---|---|
| Polyurethane as an aqueous dispersion, sold under the | 18 g AM |
| name Avalure UR 425 ® by the company Goodrich, | |
| containing 49% by weight of active material | |
| Candelilla wax | 15 g |
| Hydroxyethylcellulose | 1.9 g |
| Pigments | 5 g |
| Propylene glycol | 5 g |
| Preserving agents | qs |
| Water qs | 100 g |

Procedure

The hydroxyethylcellulose was dispersed in the presence of the preserving agents in the water at a temperature of 80° C., using a Rayneri stirrer, until a gel was formed. The polyurethane in aqueous dispersion was then added while keeping the temperature at 70° C., then the pigments, dispersed and ground beforehand in the propylene glycol using a three-roll mill, were added with stirring. The mixture was homogenized and the premolten wax was then added, with continued stirring. The mixture was cooled to room temperature (25° C.).

The composition has a film-forming polymer/fatty phase weight ratio equal to 1.2 and a dry extract of 44.9%.

The dispersing phase comprising the aqueous dispersion of polyurethane, the water and the thickener (hydroxyethylcellulose) has a viscosity of 3.9 Pa.s, measured using a Rheomat RM 180 viscometer equipped with an MS-r3 spindle.

The mascara was applied easily to the eyelashes and formed a charging makeup that is resistant to cold water and to rubbing.

### EXAMPLE 2

A mascara having the composition below was prepared:

| | |
|---|---|
| Acrylic polymer as an aqueous dispersion, sold under the | 16.5 g AM |
| name Syntran 5760 ®, containing 40% active material, by | |
| the company Interpolymer | |
| Beeswax | 20 g |
| Hydroxyethylcellulose | 1 g |
| Pigments | 5 g |
| Propylene glycol | 5 g |
| Preserving agents | qs |
| Water qs | 100 g |

The same procedure as in Example 1 was used.

The composition has a film-forming polymer/fatty phase weight ratio equal to 0.825 and a dry extract of 47.5%.

This mascara was judged as giving good thickening to the eyelashes and having good staying power.

What is claimed is:

1. A composition for coating eyelashes, comprising, in a physiologically acceptable medium:
a dispersing phase comprising an aqueous phase, at least one film-forming polymer in the form of solid particles dispersed in the aqueous phase, and at least one thickener for said aqueous phase in a sufficient amount such that the dispersing phase has a viscosity of greater than or equal to 0.2 Pa.s, and
a fatty phase with a viscosity of greater than or equal to 0.2 Pa.s, dispersed in the aqueous phase,
wherein the composition does not comprise a surfactant sufficient to disperse the fatty phase in the dispersing phase.

2. The composition according to claim 1, wherein the fatty phase comprises at least one entity chosen from waxes, gums, pasty fatty substances, semi-crystalline polymers, oils, and oils thickened with at least one structuring agent.

3. The composition according to claim 1, wherein the fatty phase comprises at least one silicone gum corresponding to the formula: 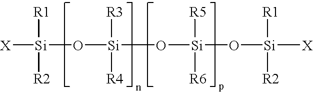
wherein:
R₁, R₂, R₅ and R₆, which may be identical or different, are chosen from alkyl radicals comprising from 1 to 6 carbon atoms,
R₃ and R₄, which may be identical or different, are chosen from alkyl radicals comprising from 1 to 6 carbon atoms and aryl radicals,
X, which may be identical or different, is chosen from alkyl radicals comprising from 1 to 6 carbon atoms, a hydroxyl radical, and a vinyl radical,
n and p, which may be identical or different, are chosen so as to give the silicone gum a viscosity of greater than 100,000 mPa.s.

4. The composition according to claim 3, wherein n and p, which may be identical or different, are chosen so as to give the silicone gum a viscosity of greater than 500,000 mPa.s.

5. The composition according to claim 3, wherein n and p, which may be identical or different, range from 0 to 5,000.

6. The composition according to claim 5, wherein n and p, which may be identical or different, range from 0 to 3,000.

7. The composition according to claim 1, wherein the fatty phase comprises at least one pasty fatty substance chosen from polydimethylsiloxanes comprising at least one pendent chain chosen from alkyl comprising from 8 to 24 carbon atoms, alkoxy comprising from 8 to 24 carbon atoms, esters of fatty alcohol comprising from 20 to 55 carbon atoms, esters of fatty acid comprising from 20 to 55 carbon atoms, polyvinyl laurate, arachidyl propionate, triisostearyl citrate, cetyl citrate, PVP/eicosene copolymer; lanolins, and derivatives thereof.

8. The composition according to claim 1, wherein the fatty phase comprises at least one semi-crystalline polymer chosen from copolymers resulting from the polymerization of at least one monomer comprising at least one crystallizable chain chosen from saturated C₁₄ to C₂₄ alkyl (meth)acrylates, C₁₁ to C₁₅ perfluoroalkyl (meth)acrylates, C₁₄ to C₂₄ N-alkyl(meth)acrylamides with or without a fluorine atom, vinyl esters comprising at least one chain chosen from C₁₄ to C₂₄ alkyl and perfluoroalkyl chains, vinyl ethers comprising at least one chain chosen from C₁₄ to C₂₄ alkyl and perfluoroalkyl chains, C₁₄ to C₂₄ alpha-olefins, para-alkylstyrenes comprising at least one alkyl group comprising from 12 to 24 carbon atoms, with at least one monomer chosen from optionally fluorinated C₁ to C₁₀ monocarboxylic acid esters and amides of the following formula: 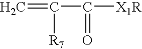
wherein R₇ is chosen from H and CH₃, R is chosen from optionally fluorinated C₁-C₁₀ alkyl groups and X₁ is chosen from O, NH and NR₈ wherein R₈ is chosen from optionally fluorinated C₁-C₁₀ alkyl groups.

9. The composition according to claim 1, wherein the fatty phase comprises at least one oil chosen from volatile and non-volatile hydrocarbon-based oils, silicone oils, and fluoro oils.

10. The composition according to claim 1, wherein the fatty phase comprises at least one oil chosen from oils thickened with at least one structuring agent.

11. The composition according to claim 10, wherein the at least one structuring agent is chosen from lipophilic gelling agents and organogelling agents.

12. The composition according to claim 10, wherein the at least one structuring agent is present in an amount ranging from 0.01% to 90% by weight relative to the weight of the fatty phase.

13. The composition according to claim 12, wherein the at least one structuring agent is present in an amount ranging from 0.5% to 80% by weight relative to the weight of the fatty phase.

14. The composition according to claim 13, wherein the at least one structuring agent is present in an amount ranging from 1% to 70% by weight relative to the weight of the fatty phase.

15. The composition according to claim 1, wherein the fatty phase comprises at least one wax chosen from beeswax, lanolin wax, Chinese insect waxes, candelilla wax, ouricurry wax, cork fibre wax, sugarcane wax, berry wax, Japan wax, sumach wax, montan wax, waxy copolymers and esters thereof, waxes obtained by catalytic hydrogenation of animal or plant oils comprising at least one fatty chain chosen from C₈-C₃₂ linear and branched fatty chains, silicone waxes and fluoro waxes.

16. The composition according to claim 1, wherein the fatty phase comprises at least one wax chosen from waxes with a melting point ranging from 30° C. to 70° C.

17. The composition according to claim 1, wherein the fatty phase comprises at least one polar wax.

18. The composition according to claim 1, wherein the fatty phase comprises at least one wax chosen from candelilla wax, beeswax, berry wax, hydrogenated jojoba wax, and olive wax obtained by hydrogenation of olive oil esterified with stearyl alcohol.

19. A composition for coating eyelashes, comprising, in a physiologically acceptable medium:
a dispersing phase comprising an aqueous phase, at least one film-forming polymer in the form of solid particles dispersed in the aqueous phase, and at least one thickener for said aqueous phase in a sufficient amount such that the aqueous phase has a viscosity of greater than or equal to 0.2 Pa.s, and
at least one wax in the form of particles dispersed in the aqueous phase, wherein the composition does not comprise any surfactant sufficient to disperse the at least one wax in the aqueous phase.

20. The composition according to claim 19, wherein the at least one wax is chosen from beeswax, lanolin wax, Chinese insect waxes, candelilla wax, ouricurry wax, cork fibre wax, sugarcane wax, berry wax, Japan wax, sumach wax, montan wax, waxy copolymers and esters thereof, waxes obtained by catalytic hydrogenation of animal or plant oils comprising at least one fatty chain chosen from C₈-C₃₂ linear and branched fatty chains, silicone waxes and fluoro waxes.

21. The composition according to claim 19, wherein the at least one wax is chosen from waxes with a melting point ranging from 30° C. to 70° C.

22. The composition according to claim 19, wherein the at least one wax is chosen from polar waxes.

23. The composition according to claim 19, wherein the at least one wax is chosen from candelilla wax, beeswax, berry wax, hydrogenated jojoba wax, and olive wax obtained by hydrogenation of olive oil esterified with stearyl alcohol.

24. The composition according to claim 1, wherein the fatty phase is present in an amount ranging from 5% to 60% by weight, relative to the total weight of the composition.

25. The composition according to claim 24, wherein the fatty phase is present in an amount ranging from 10% to 50% by weight, relative to the total weight of the composition.

26. The composition according to claim 25, wherein the fatty phase is present in an amount ranging from 15% to 40% by weight, relative to the total weight of the composition.

27. The composition according to claim 1, wherein the particles of the at least one film-forming polymer have a particle size ranging from 5 nm to 600 nm.

28. The composition according to claim 27, wherein the particles of the at least one film-forming polymer have a particle size ranging from 20 nm to 300 nm.

29. The composition according to claim 1, wherein the at least one film-forming polymer is chosen from vinyl polymers, polyurethanes, polyesters, polyesteramide polymers and alkyds.

30. The composition according to claim 1, wherein the at least one film-forming polymer is chosen from hydrophobic film-forming polymers.

31. The composition according to claim 1, wherein the at least one film-forming polymer is present in a solids content ranging from 1% to 60% by weight, relative to the total weight of the composition.

32. The composition according to claim 31, wherein the at least one film-forming polymer is present in a solids content ranging from 5% to 40% by weight, relative to the total weight of the composition.

33. The composition according to claim 32, wherein the at least one film-forming polymer is present in a solids content ranging from 10% to 30% by weight, relative to the total weight of the composition.

34. The composition according to claim 1, wherein the aqueous phase is present in an amount ranging from 5% to 95% by weight relative to the total weight of the composition.

35. The composition according to claim 1, wherein the dispersing phase has a viscosity ranging from 0.2 Pa.s to 50 Pa.s.

36. The composition according to claim 1, wherein the at least one thickener for the aqueous phase is chosen from water-soluble cellulose-based thickeners, guar gums, quaternized guar gums, nonionic guar gums, xanthan gum, carob gum, scleroglucan gum, gellan gum, rhamsan gum, karaya gum, alginates, maltodextrin, starch and its derivatives, hyaluronic acid and its salts, clays, crosslinked polyacrylic acids, polglyceryl (meth)acrylate polymers, polyvinylpyrrolidone, polyvinyl alcohol, crosslinked acrylamide polymers and copolymers, crosslinked methacryloyloxyethyltrimethylammonium chloride homopolymers, and associative polymers.

37. The composition according to claim 1, wherein the at least one thickener for the aqueous phase is present in an amount ranging from 0.1% to 15% by weight, relative to the total weight of the composition.

38. The composition according to claim 37, wherein the at least one thickener for the aqueous phase is present in an amount ranging from 1% to 10% by weight, relative to the total weight of the composition.

39. The composition according to claim 38, wherein the at least one thickener for the aqueous phase is present in amount ranging from 1% to 5% by weight, relative to the total weight of the composition.

40. The composition according to claim 1, wherein the solids content of the composition ranges from 30% to 60% by weight, relative to the total weight of the composition.

41. The composition according to claim 40, wherein the solids content of the composition ranges from 35% to 55% by weight, relative to the total weight of the composition.

42. The composition according to claim 41, wherein the solids content of the composition ranges from 40% to 50% by weight, relative to the total weight of the composition.

43. The composition according to claim 1, wherein the composition is a mascara composition.

44. A non-therapeutic cosmetic care and/or makeup process for a keratin material, comprising applying to the keratin material a composition comprising, in a physiologically acceptable medium:
a dispersing phase comprising an aqueous phase, at least one film-forming polymer in the form of solid particles dispersed in the aqueous phase, and at least one thickener for said aqueous phase in a sufficient amount such that the dispersing phase has a viscosity of greater than or equal to 0.2 Pa.s, and
a fatty phase with a viscosity of greater than or equal to 0.2 Pa.s, dispersed in the aqueous phase,
wherein the composition does not comprise a surfactant sufficient to disperse the fatty phase in the dispersing phase.

45. A non-therapeutic cosmetic care and/or makeup process for a keratin material, comprising applying to the keratin material a composition comprising, in a physiologically acceptable medium:
a dispersing phase comprising an aqueous phase, at least one film-forming polymer in the form of solid particles dispersed in the aqueous phase, and at least one thickener for said aqueous phase in a sufficient amount such that the aqueous phase has a viscosity of greater than or equal to 0.2 Pa.s, and
at least one wax in the form of particles dispersed in the aqueous phase,
wherein the composition does not comprise any surfactant sufficient to disperse the at least one wax in the aqueous phase.

46. A method for coating keratin fibres, comprising applying to the keratin fibres a cosmetic composition comprising, in a physiologically acceptable medium,
a dispersing phase comprising an aqueous phase, at least one film-forming polymer in the form of solid particles dispersed in the aqueous phase, and at least one thickener for said aqueous phase in a sufficient amount such that the dispersing phase has a viscosity of greater than or equal to 0.2 Pa.s, and
a fatty phase with a viscosity of greater than or equal to 0.2 Pa.s dispersed in the aqueous phase,
wherein
the composition does not comprise a surfactant sufficient to disperse the fatty phase in the dispersing phase, and
the composition is effective for obtaining a smooth and uniform film deposited on the keratin fibres and/or a film having at least one of the following properties: being resistant to at least one of water, tears, and perspiration and having a charging effect on the keratin fibres.

47. A method for coating keratin fibres, comprising applying to the keratin fibres a cosmetic composition comprising, in a physiologically acceptable medium,
a dispersing phase comprising an aqueous phase, at least one film-forming polymer in the form of solid particles dispersed in the aqueous phase, and at least one thickener for said aqueous phase in a sufficient amount such that the aqueous phase has a viscosity of greater than or equal to 0.2 Pa.s, and
at least one wax in the form of particles dispersed in the aqueous phase,
wherein
the composition does not comprise a surfactant sufficient to disperse the fatty phase in the dispersing phase, and
the composition is effective for obtaining a smooth and uniform film deposited on the keratin fibres and/or a film having at least one of the following properties: being resistant to at least one of water, tears, and perspiration and having a charging effect on the keratin fibres.

48. A composition for coating the eyelashes, comprising, in a physiologically acceptable medium:
a dispersing phase comprising an aqueous phase, at least one film-forming polymer in the form of solid particles dispersed in the aqueous phase, and at least one thickener for the said aqueous phase in a sufficient amount such that the dispersing phase has a viscosity of greater than or equal to 0.2 Pa.s, and
a fatty phase with a viscosity of greater than or equal to 0.2 Pa.s, dispersed in the aqueous phase,
wherein the composition does not comprise a surfactant to disperse the fatty phase in the dispersing phase.
